(19) United States
(12) Patent Application Publication
(10) Pub. No.: US 2013/0045170 A1
(43) Pub. Date: Feb. 21, 2013

(54) IMMUNITY BOOSTING GUM

(71) Applicant: Terence O'Neil, Newport Coast, CA (US)

(72) Inventor: Terence O'Neil, Newport Coast, CA (US)

(21) Appl. No.: 13/564,554

(22) Filed: Aug. 1, 2012

Related U.S. Application Data
(60) Provisional application No. 61/525,612, filed on Aug. 19, 2011.

(51) Int. Cl. A61K 9/68; A61P 37/04

(52) U.S. Cl. 424/48

(57) ABSTRACT

A chewing gum is disclosed with immunity boosting properties to ward off illness and promote favorable health. The chewing gum begins with a gum base, to which various medicinal elements are added so that they can be released during mastication of the gum. The medicinal elements include zinc, Vitamin C, elderberry, astragalus, eleutherococcus, and echinacea.

### CROSS-REFERENCES TO RELATED APPLICATIONS

This application claims priority from U.S. Provisional Application No. 61/525,612, filed Aug. 19, 2011 incorporated by reference in its entirety.

### BACKGROUND

There are many gums on the market that target different customer groups, from children to tobacco users to halitosis sufferers. Most of these gums offer some flavor to enhance the experience, and some are made more pliable to promote bubble blowing. A particular type of gum is referred to as “functional gum,” because it has some medicinal effect or contributes to one's health in some way. For example, nicotine gums release nicotine into the body to help stave off the effects of tobacco withdrawal. Other gums release caffeine that gives the body an energy boost. But the present inventor is unaware of any gum that specifically targets the body's immune system. The present invention is a chewing gum that can support and boost this essential body function.

### SUMMARY OF THE INVENTION

The present invention is a chewing gum that can promote the resistance of the onset of illness by incorporating ingredients known to fight disease and other maladies. In particular, the ingredients of this gum include Zinc, Vitamin C, Elderberry Extract, Eleutherococcus Extract (Siberian Ginseng), Astragalus, and Echinacea. Each ingredient will be discussed accordingly.

Zinc:

Zinc is considered an essential mineral with wound healing, antioxidant, and immunity functions. Zinc is generally thought to be necessary to a healthy immune system. It has been hypothesized that zinc blocks the binding of Rhinovirus (a common cold virus) to the ICAM-1 receptor, thereby reducing the duration and severity of cold symptoms. Medical experts report that zinc given within 24 hours of onset of cold symptoms reduces the duration and severity of a cold. Furthermore, it was found that supplementation with zinc for at least 5 months decreased colds, and school absenteeism in children. A zinc deficiency can impair immune cells, such as macrophages, neutrophils, B-Cells, and natural killer cells, and Zinc is required to activate T-lymphocytes.

Vitamin C:

Vitamin C was made famous by the Nobel Prize Winner Linus Pauling. He believed that Vitamin C reduced the incidence of colds. There is some evidence to support the view that daily Vitamin C, prior to having a cold, may reduce the duration of cold episodes. Other studies have shown a more substantial reduction in duration of cold episodes in athletes, and those exposed to cold. Supplementation with Vitamin C was found to improve components of the immune system, such as lymphocytes, anti-bacterial, and natural killer cell activities. Vitamin C also is an antioxidant, protecting the body from free radicals, which may damage one's DNA.

Elderberry:

Elderberry has been used for many years to treat wounds and colds. In Germany, the flower is used to treat colds and flu. Elderberry contains flavonoids, which are excellent antioxidants. Elderberry extract was shown to activate the immune system by stimulating a broad range of cytokine messengers. There is also evidence to support that Elderberry reduces the duration of Flu symptoms.

Eleutherococcus Extract (Siberian Ginseng):

Eleuthero (Siberian Ginseng) is an adaptogen that has been used for hundreds of years in China and Russia. An adaptogen is an herb that is thought to bring the body back into balance, and help it cope with mental or physical stress better. It has been used in Russia and China to improve energy and immune function. A study by Kan Jang of the Swedish Herbal institute showed that Siberian Ginseng plus andographis improved symptoms of the common cold when started within 72 hours of symptom onset. See, e.g., http://www.nlm.nih.gov/medlineplus/druginfo/natural/985.html

Astragalus:

Astragalus is an adaptogen that has been used in Chinese medicine for thousands of years. An adaptogen is an herb that is thought to bring the body back into balance, and help it cope with mental or physical stress better. In Chinese medicine, Astragalus is used to protect and support the immune system. Astragalus was shown in a lab to activate immune cells, such as B-Cells and Macrophages. It is also an antioxidant, protecting the body from free radicals.

Echinacea:

Echinacea is herb that was used by Native Americans for hundreds of years to treat infections. It is still very popular in the U.S. and Germany. Some studies have shown Echinacea to activate the immune system, and work as an antioxidant. A recent meta-analysis showed a 58% decrease in the odds of developing the common cold. Echinacea has also been found to stimulate immune cells in a laboratory.

In conclusion, this unique combination of ingredients, coupled with the fact that the product is in gum form, solidifies its difference from other products on the market.

### DETAILED DESCRIPTION OF THE PREFERRED EMBODIMENTS

The present invention is a chewing gum that includes and releases into the body orally a number of medicinal elements as the gum is chewed. It begins with a gum base, and then other ingredients are added to the product to achieve the final gum. Chewing gum base consists either of natural latex or a synthetic substitute. Natural latex such as chicle is harvested by making large X-marks on rubber trees and then collecting the substance as it runs down the tree. Gum bases are non-nutritive masticatory substances as defined by the US FDA “Code of Federal Regulation”. They are inert and insoluble non-nutritive products used as a support for the edible and soluble portion of the chewing gum (sugar, glucose, polyols and flavors). Gum bases are produced through a blend of raw materials that can be categorized in five classes:

1. Elastomers act as the key ingredient and provide elasticity

2. Resins act as binders and softeners

3. Plasticizers render the elastomer soft to ensure thorough blending of the gum base

4. Fillers contribute to the overall texture

5. Antioxidants prevent oxidation of the gum base and flavors during shelf life

The ingredients of gums are typically divided into soluble and insoluble parts. The insoluble components are the gum base and some flavoring material; all other ingredients—like sugar, glucose syrup, or polyols and intensive sweeteners in sugar-free products—are water soluble. A wide variety of gum bases are known in the art. They can be combined with acid or non-acid flavoring, sugar or sugar substitutes as well as vitamins or active ingredient in functional and pharmaceutical products. Products can be made in various formats depending on the available process technology.

The process by which the gum is made is set forth below in four steps.

Step 1: The natural and/or artificial gum bases are first ground into a coarse meal and mixed to ensure uniform consistency. The blend is then placed in a warm room to dry for up to two days. During drying, hot air is continually passed over the mixture.

Step 2: Next, the gum base is cooked in kettles at approximately 243 degrees Fahrenheit (116 degrees Celsius) until it has melted into a thick syrup. To purify it, it is passed through screens and placed it in a high speed centrifuge before re-filtering it. Then it filtered through finer screens.

Step 3: The dried and filtered gum base is now ready for additives. It is placed in kettles to be cooked, and additional ingredients are stirred in by large steel blades. First, extremely fine powdered sugar and corn syrup are added, or artificial sweeteners. Flavorings are added next, followed by softeners. Then vitamin C, Zinc, Astragalus, Elderberry, Eleutherococcus, Echinacea are slowly added to the mixture. When the mixture is thoroughly mixed and smooth, it is rolled out onto belts and cooled by being exposed to cold air.

Step 4: The next step is kneading. For several hours, machines gently pummel or “knead” the mass until it is properly smooth and rubbery, and then it is put on a rolling slab and reduced to the proper thickness. After being coated with an exterior of xylitol, the gum is scored into a pattern of rectangles, or circles, and broken into pieces of gum. The gum is now ready to be packaged and shipped to retail outlets.

The present invention has identified several important combinations of medicinal elements that make up the immunity gum of the present invention. These combinations include the following:

1. zinc, elderberry, astragalus, eleutherococcus

2. Elderberry, astragalus, eleutherococcus

3. zinc, elderberry

4. zinc, astragalus

5. vitamin C, elderberry

6. vitamin C, elderberry, astragalus

7. vitamin C, zinc, elderberry

8. zinc, vitamin C, elderberry, astragalus, eleutherococcus, echinacea

9. vitamin C, elderberry, astragalus, eleutherococcus

10. vitamin C, elderberry, astragalus, echinacea, eleutherococcus

11. vitamin C, zinc

12. vitamin C, zinc, elderberry, astragalus

13. vitamin C, zinc, elderberry, echinacea

14. vitamin C, zinc, elderberry, astragalus, echinacea

15. Echinacea, vitamin C

16. Echinacea, zinc

17. Echinacea, elderberry, vitamin C

18. Echinacea, elderberry, zinc

19. Echinacea, elderberry, zinc, vitamin C

While the quantities of the aforementioned medicinal elements can vary, in the preferred embodiments the quantities are set forth as follows:

Zinc: 1 mg to 60 mg

Vitamin C: 1 mg to 1500 mg

Astragalus: 0.001 mg to 300 mg

Elderberry: 0.001 mg to 500 mg

Eleutherococcus: 0.001 mg to 300 mg

Echinacea: 0.001 mg-500 mg

The gum of the present invention provides relief and protection against various illnesses, and is not known in the prior art. The invention should not be limited by any of the examples above, but rather by the appended claims below.

I claim:

1. An immunity boosting chewing gum comprising:
a gum base; and
two immunity boosting ingredients selected from a group comprising zinc, elderberry, and astragalus.

2. The immunity boosting chewing gum of claim 1, further comprising Vitamin C.

3. The immunity boosting chewing gum of claim 1, further comprising eleutherococcus.

4. The immunity boosting chewing gum of claim 1, further comprising echinacea.

5. An immunity boosting chewing gum comprising:
a gum base; and
two immunity boosting ingredients selected from a group comprising zinc, elderberry, and Vitamin C.

6. The immunity boosting chewing gum of claim 5, further comprising echinacea.

7. The immunity boosting chewing gum of claim 5, further comprising astragalus.

8. An immunity boosting chewing gum comprising:
a gum base; and
three immunity boosting ingredients selected from a group comprising echinacea, zinc, elderberry, and Vitamin C.

9. The immunity boosting chewing gum of claim 8, further comprising eleutherococcus.

10. The immunity boosting chewing gum of claim 8, further comprising astragalus.
